(19) United States
(12) Patent Application Publication
(10) Pub. No.: US 2024/0000769 A1
(43) Pub. Date: Jan. 4, 2024

(54) AMORPHOUS SOLID DISPERSIONS

(71) Applicant: UCB Biopharma SRL, Brussels (BE)

(72) Inventors: Faiza Laredj, Brussels (BE); Rosanna Chirico, Brussels (BE); Aymeric Ousset, Brussels (BE)

(21) Appl. No.: 18/267,925

(22) PCT Filed: Dec. 16, 2021
(86) PCT No.: PCT/EP2021/086061

§ 371 Date: Jun. 16, 2023

(30) Foreign Application Priority Data
Dec. 18, 2020 (EP) 20215253.4

(51) Int. Cl. A61K 31/4725; A61K 9/20; A61K 9/28; A61K 9/16

(52) U.S. Cl. CPC A61K 31/4725; A61K 9/2004; A61K 9/28; A61K 9/1682

(57) ABSTRACT

The present invention relates to solid dispersions of amorphous compound of formula (I), and a polymer matrix, their processes of preparation and their uses in therapy.

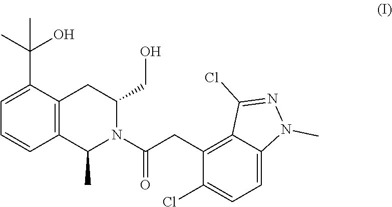 [SMILES: C[C@H]1c2cccc(C(C)(C)O)c2C[C@H](CO)N1C(=O)Cc1c(Cl)ccc2c1c(Cl)nn2C]

### FIELD OF THE INVENTION

This invention relates to solid dispersions of amorphous 2-(3,5-dichloro-1-methyl-indazol-4-yl)-1-[(1S,3R)-3-(hydroxymethyl)-5-(1-hydroxy-1-methyl-ethyl)-1-methyl-3,4-dihydro-1H-isoquinolin-2-yl]ethanone.

This invention also relates to processes for the preparation of these amorphous solid dispersions and to pharmaceutical compositions including such dispersions.

### BACKGROUND OF THE INVENTION

International patent application n° PCT/EP2020/068183, published as WO2021/001288, discloses 2-(3,5-dichloro-1-methyl-indazol-4-yl)-1-[(1S,3R)-3-(hydroxymethyl)-5-(1-hydroxy-1-methyl-ethyl)-1-methyl-3,4-dihydro-1H-isoquinolin-2-yl]ethanone of formula (I),

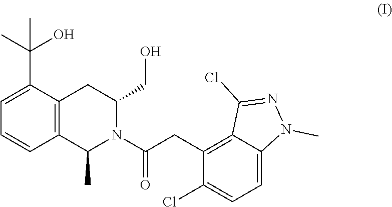 [SMILES: C[C@H]1c2cccc(C(C)(C)O)c2C[C@H](CO)N1C(=O)Cc1c(Cl)ccc2c1c(Cl)nn2C]

which compound acts as a D1 Positive Allosteric Modulator and is accordingly of benefit as a pharmaceutical agent for the treatment of diseases in which D1 receptors play a role.

International patent application n° PCT/EP2020/068183, published as WO2021/001288, further discloses that compound of formula (I) may be useful in the treatment and/or prevention of cognitive and negative symptoms in schizophrenia, cognitive impairment related to neuroleptic therapy, Mild Cognitive impairment (MCI), impulsivity, Attention-Defficit Hyperactivity Disorder (ADHD), Parkinson's disease and other movement disorders, dystonia, Parkinson's dementia, Huntington's disease, dementia with Lewy Body, Alzheimer's disease drug addiction, sleep disorders, apathy, traumatic spinal cord injury or neuropathic pain.

It is therefore desirable to develop formulations of compound of formula (I) that will be suitable for administration to patients suffering from any one of the above-mentioned disease.

In particular, Example 2.8. of international patent application n° PCT/EP2020/068183, published as WO2021/001288, discloses inter alia a monohydrated crystalline form of of 2-(3,5-dichloro-1-methyl-indazol-4-yl)-1-[(1S,3R)-3-(hydroxymethyl)-5-(1-hydroxy-1-methyl-ethyl)-1-methyl-3,4-dihydro-1H-isoquinolin-2-yl]ethanone.

This monohydrated crystalline form of 2-(3,5-dichloro-1-methyl-indazol-4-yl)-1-[(1S,3R)-3-(hydroxymethyl)-5-(1-hydroxy-1-methyl-ethyl)-1-methyl-3,4-dihydro-1H-isoquinolin-2-yl]ethanone has limited solubility which may result in difficulties to formulate it and/or low biovailability, if an oral administration is desired.

There is therefore a need to improve the solubility of the monohydrated crystalline form of 2-(3,5-dichloro-1-methyl-indazol-4-yl)-1-[(1S,3R)-3-(hydroxymethyl)-5-(1-hydroxy-1-methyl-ethyl)-1-methyl-3,4-dihydro-1H-isoquinolin-2-yl]ethanone so that it can be incorporated in a pharmaceutical composition, in particular for oral administration.

### SUMMARY OF THE INVENTION

The present invention provides amorphous solid dispersions of 2-(3,5-dichloro-1-methyl-indazol-4-yl)-1-[(1S,3R)-3-(hydroxymethyl)-5-(1-hydroxy-1-methyl-ethyl)-1-methyl-3,4-dihydro-1H-isoquinolin-2-yl]ethanone of formula (I).

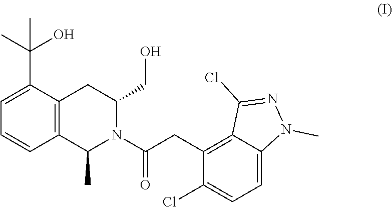 [SMILES: C[C@H]1c2cccc(C(C)(C)O)c2C[C@H](CO)N1C(=O)Cc1c(Cl)ccc2c1c(Cl)nn2C]

In another aspect the present invention provides processes for the preparation of amorphous solid dispersions of 2-(3,5-dichloro-1-methyl-indazol-4-yl)-1-[(1S,3R)-3-(hydroxymethyl)-5-(1-hydroxy-1-methyl-ethyl)-1-methyl-3,4-dihydro-1H-isoquinolin-2-yl]ethanone of formula (I).

In a further aspect, the present invention provides a pharmaceutical composition comprising an amorphous solid dispersion of 2-(3,5-dichloro-1-methyl-indazol-4-yl)-1-[(1S,3R)-3-(hydroxymethyl)-5-(1-hydroxy-1-methyl-ethyl)-1-methyl-3,4-dihydro-1H-isoquinolin-2-yl]ethanone of formula (I).

In yet a further aspect, the present invention provides such amorphous solid dispersions of 2-(3,5-dichloro-1-methyl-indazol-4-yl)-1-[(1S,3R)-3-(hydroxymethyl)-5-(1-hydroxy-1-methyl-ethyl)-1-methyl-3,4-dihydro-1H-isoquinolin-2-yl]ethanone of formula (I), or pharmaceutical composition thereof, for use in the treatment and/or prevention of cognitive and negative symptoms in schizophrenia, cognitive impairment related to neuroleptic therapy, Mild Cognitive impairment (MCI), impulsivity, Attention-Defficit Hyperactivity Disorder (ADHD), Parkinson's disease and other movement disorders, dystonia, Parkinson's dementia, Huntington's disease, dementia with Lewy Body, Alzheimer's disease drug addiction, sleep disorders, apathy, traumatic spinal cord injury or neuropathic pain.

### DESCRIPTION OF THE DRAWINGS

FIG. 1 represents an X-Ray Powder Diffraction Pattern of amorphous solid dispersion ASD1 as further described in Example 3.1.

FIG. 2 represents an X-Ray Powder Diffraction Pattern of amorphous solid dispersion ASD2 as further described in Example 3.1.

FIG. 3 represents an X-Ray Powder Diffraction Pattern of amorphous solid dispersion ASD3 as further described in Example 3.1.

FIG. 4 represents an X-Ray Powder Diffraction Pattern of amorphous solid dispersion ASD4 as further described in Example 3.1.

FIG. 5 represents an X-Ray Powder Diffraction Pattern of amorphous solid dispersion ASD1 as described in Example 3, after 12 months at room temperature.

FIG. 6 represents X-Ray Powder Diffraction Patterns of amorphous solid dispersions ASD2, ASD3 and ASD4 as described in Example 3 after 10 months at room temperature.

FIG. 7 represents the dissolution profile over time of amorphous solid dispersion ASD1 into compound of formula (I), as further described in Example 5.

FIG. 8 represents the dissolution profile over time of the monohydrated crystalline form of 2-(3,5-dichloro-1-methyl-indazol-4-yl)-1-[(1S,3R)-3-(hydroxymethyl)-5-(1-hydroxy-1-methyl-ethyl)-1-methyl-3,4-dihydro-1H-isoquinolin-2-yl]ethanone (compound of formula (Ia)) as further described in Example 5.

FIG. 9 represents an X-Ray Powder Diffraction Pattern of amorphous solid dispersion ASD5 as further described in Example 3.2.

FIG. 10 represents an X-Ray Powder Diffraction Pattern of amorphous solid dispersion ASD6 as further described in Example 3.2.

FIG. 11 represents an X-Ray Powder Diffraction Pattern of amorphous solid dispersion ASD7 as further described in Example 3.2.

FIG. 12 represents an X-Ray Powder Diffraction Pattern of amorphous solid dispersion ASD8 as further described in Example 3.2.

FIG. 13 represents an X-Ray Powder Diffraction Pattern of amorphous solid dispersion ASD9 as further described in Example 3.2.

FIG. 14 represents an X-Ray Powder Diffraction Pattern of amorphous solid dispersion ASD10 as further described in Example 3.2.

FIG. 15 represents an X-Ray Powder Diffraction Pattern of amorphous solid dispersion ASD11 as further described in Example 3.2.

FIG. 16 represents an X-Ray Powder Diffraction Pattern of amorphous solid dispersion ASD12 as further described in Example 3.2.

FIG. 17 represents an X-Ray Powder Diffraction Pattern of amorphous solid dispersion ASD13 as further described in Example 3.2.

FIG. 18 represents an X-Ray Powder Diffraction Pattern of amorphous solid dispersion ASD14 as further described in Example 3.2.

FIG. 19 represents an X-Ray Powder Diffraction Pattern of amorphous solid dispersion ASD15 as further described in Example 3.2.

FIG. 20 represents bioavailability of compound of formula (I) as a function of the administered dose of a suspension of ASD1 prepared according to Example 6.1.

FIG. 21 represents an X-Ray Powder Diffraction Pattern of amorphous solid dispersion ASD16 as further described in Example 3.2.

FIG. 22 represents an X-Ray Powder Diffraction Pattern of amorphous solid dispersion ASD17 as further described in Example 3.2.

FIG. 23 represents the dissolution profile over time of Tablet A into compound of formula (I), as further described in Example 7.2.

FIG. 24 represents the dissolution profile over time of Tablet B into compound of formula (I), as further described in Example 7.2

FIG. 25 represents the dissolution profile over time of Tablet C into compound of formula (I), as further described in Example 7.2.

FIG. 26 represents the dissolution profile over time of Tablet D into compound of formula (I), as further described in Example 7.2.

FIG. 27 represents the X-Ray Powder Diffraction Patterns of Tablets A before and after 12 months of storage according to the conditions described in Example 7.3.

FIG. 28 represents the X-Ray Powder Diffraction Patterns of Tablets B before and after 12 months of storage according to the conditions described in Example 7.3.

FIG. 29 represents the X-Ray Powder Diffraction Patterns of Tablets D before and after 12 months of storage according to the conditions described in Example 7.3.

### DETAILED DESCRIPTION OF THE INVENTION

As used herein, the term “amorphous solid dispersion” refers to a solid dispersion comprising amorphous compound of formula (I) and a polymer matrix as defined herein.

As used herein, the term “solid dispersion” refers to a system in a solid state comprising at least two components wherein one component is dispersed throughout the other component.

As used herein “amorphous compound of formula (I)” means compound of formula (I) essentially free of crystalline form. The amorphous nature of a solid is generally determined by X-Ray Powder Diffraction (XRPD). The X-ray Powder Diffraction Pattern of an amorphous solid generally represent broad halos devoid of sharp peaks, as will be apparent to the person skilled in the art using conventional XRPD techniques.

“Essentially free of crystalline form” means in relation to compound of formula (I) that it contains at least 95%, suitably at least about 98%, ideally at least about 99% of compound of formula (I) in amorphous form as measured by X-Ray Powder Diffraction according to conventional methods as further described herein.

The term “polymer matrix” as used herein refers to any one of the polymers selected from the group consisting of hydroxy propyl methyl cellulose acetate succinate (also referred to as HPCMAS), co-polymer N-vinyl-2-pyrrolidone/vinyl acetate (also referred to as PVPVA), polyvinyl pyrrolidone (also referred to as PVP), hypromellose phtalate (also referred to as HPMCP), hypromellose (also referred to as HPMC). These polymer matrices are generally commercially available and are available in different physical/chemical grade types as will be apparent from the experimental section.

In a first aspect, the present invention provides a solid dispersion comprising amorphous compound of formula (I) and hydroxy propyl methyl cellulose acetate succinate.

In a second aspect, the present invention provides a solid dispersion comprising amorphous compound of formula (I) and co-polymer N-vinyl-2-pyrrolidone/vinyl acetate.

In a third aspect, the present invention provides a solid dispersion comprising amorphous compound of formula (I) and polyvinyl pyrrolidone.

In a fourth aspect, the present invention provides a solid dispersion comprising amorphous compound of formula (I) and hypromellose phtalate.

In a fifth aspect, the present invention provides a solid dispersion comprising amorphous compound of formula (I) and hypromellose.

The amorphous solid dispersion according to the present invention comprises about 30% to about 60% weight of amorphous compound of formula (I) compared to total weight of amorphous solid dispersion, herein after referred to as “weight %”.

In a first embodiment according to the present invention, the amorphous solid dispersion comprises about 30 weight % of amorphous compound of formula (I).

In a second embodiment according to the present invention, the amorphous solid dispersion comprises about 40 weight % of amorphous compound of formula (I).

In a third embodiment according to the present invention, the amorphous solid dispersion comprises about 50 weight % of amorphous compound of formula (I).

In a fourth embodiment according to the present invention, the amorphous solid dispersion comprises about 60 weight % of amorphous compound of formula (I).

Particular examples of amorphous solid dispersions according to the present invention, include an amorphous solid dispersion comprising about 30 weight % of compound of formula (I) with hydroxy propyl methyl cellulose acetate succinate; an amorphous solid dispersion comprising about 40 weight % of compound of formula (I) with hydroxy propyl methyl cellulose acetate succinate; an amorphous solid dispersion comprising about 50 weight % of compound of formula (I) with hydroxy propyl methyl cellulose acetate succinate; an amorphous solid dispersion comprising about 60 weight % of compound of formula (I) with hydroxy propyl methyl cellulose acetate succinate; an amorphous solid dispersion comprising about 40 weight % of compound of formula (I) with hypromellose; an amorphous solid dispersion comprising about 50 weight % of compound of formula (I) with hypromellose; an amorphous solid dispersion comprising about 40 weight % of compound of formula (I) with hypromellose phthalate; an amorphous solid dispersion comprising about 50 weight % of compound of formula (I) with hypromellose phthalate; an amorphous solid dispersion comprising about 40 weight % of compound of formula (I) with polyvinyl pyrrolidone; an amorphous solid dispersion comprising about 50 weight % of compound of formula (I) with polyvinyl pyrrolidone; and an amorphous solid dispersion comprising about 40 weight % of compound of formula (I) with co-polymer N-vinyl-2-pyrrolidone/vinyl acetate.

The amorphous solid dispersions according to the present invention may, for example, be prepared by spray drying. Typically, the monohydrated crystalline form of of 2-(3,5-dichloro-1-methyl-indazol-4-yl)-1-[(1S,3R)-3-(hydroxymethyl)-5-(1-hydroxy-1-methyl-ethyl)-1-methyl-3,4-dihydro-1H-isoquinolin-2-yl]ethanone, herein after referred to as compound of formula (Ia), and the polymer matrix as defined herein, are dissolved in a suitable solvent, or a mixture of suitable solvents, to form a feed solution and thereafter the feed solution is spray dried to form the amorphous solid dispersion as a powder. Spray-drying is a process well known to the person skilled in the art preparing amorphous solid dispersions.

The spray drying process according to the present invention typically runs continuously and comprises the following steps: (i) preparation of feed solution where compound of formula (Ia) and carrier are dissolved into organic solvent(s), (ii) conveying the feed solution through an atomizer into a drying chamber, (iii) contacting the droplets formed in step (ii) with a hot drying gas, (iv) evaporating the solvent and (v) separating the dried solid particles from the drying gas.

Suitable solvents for the spray drying process according to the present invention are dichloromethane (DCM), methanol, ethanol, ethyl acetate, acetone, water or mixtures thereof. Particular solvent used according to the present invention is a mixture of dichloromethane and methanol as further described in the Examples.

Atomization is generally performed by conventional means, for example, by feeding the solution through nozzles at a pressure comprised between about 0.5 bar and about 2.5 bar, ideally between about 1.00 bar and about 2.5 bar.

The hot drying gas used in the drying chamber may be selected from air, nitrogen enriched air, or argon. The temperature of the hot drying gas is generally comprised between about and about 120° C., suitably between about 60° C. and 120° C., resulting in an outlet temperature comprised between about 40° C. and about 65° C.

The solid particles obtained after step (v) may be further dried by conventional means at a temperature comprised between about 25° C. and about 50° C., either at atmospheric or reduced pressure.

Alternatively, amorphous solid dispersions may be prepared by a process involving hot melt extrusion. A hot melt extrusion process generally comprises: i) a feeding a system containing the material to be extruded, in this case the powder mixture of compound of formula (Ia) and the polymer matrix, in a continuous flow or in a controlled manner; ii) conveying section made of barrel and screw(s) that aims to transport, melt and homogenously mix the feeded blend; iii) shaping the melt in the desired form including sheet, film or strand dies; iv) further steps of downstream processes including cooling, possibly pelletizing or milling, and collecting the resulting amorphous solid dispersions.

The hot melt extrusion process is generally conducted at a barrel temperature greater than 100° C., suitably at a temperature greater than 150° C.

Thus, in another aspect, the present invention provides a process for the preparation of amorphous solid dispersions of compound of formula (I) by spray-drying or hot melt extrusion.

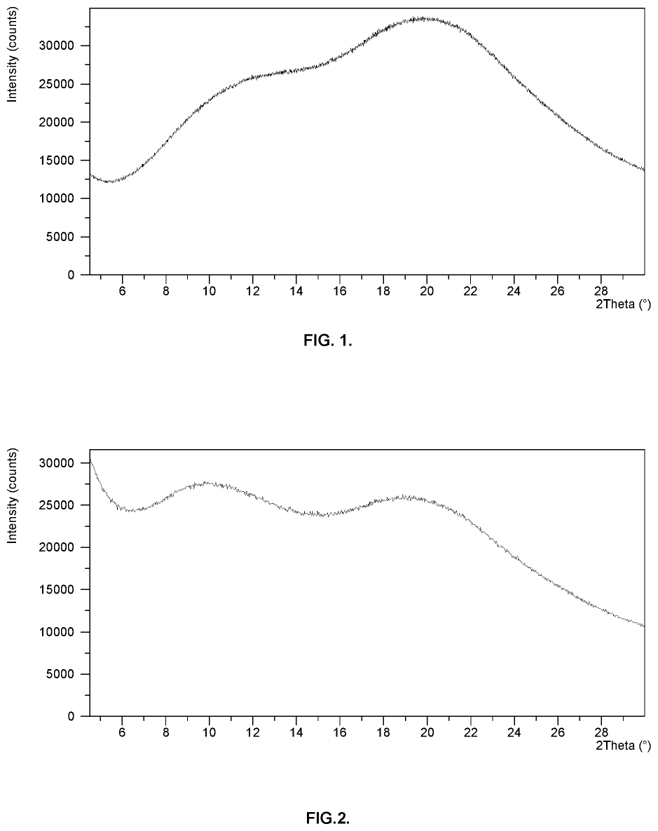

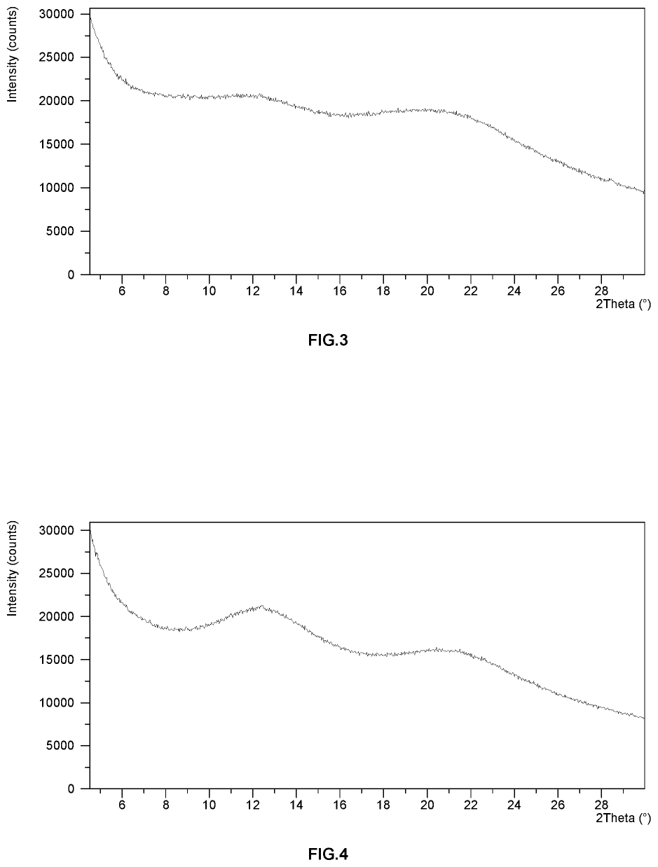

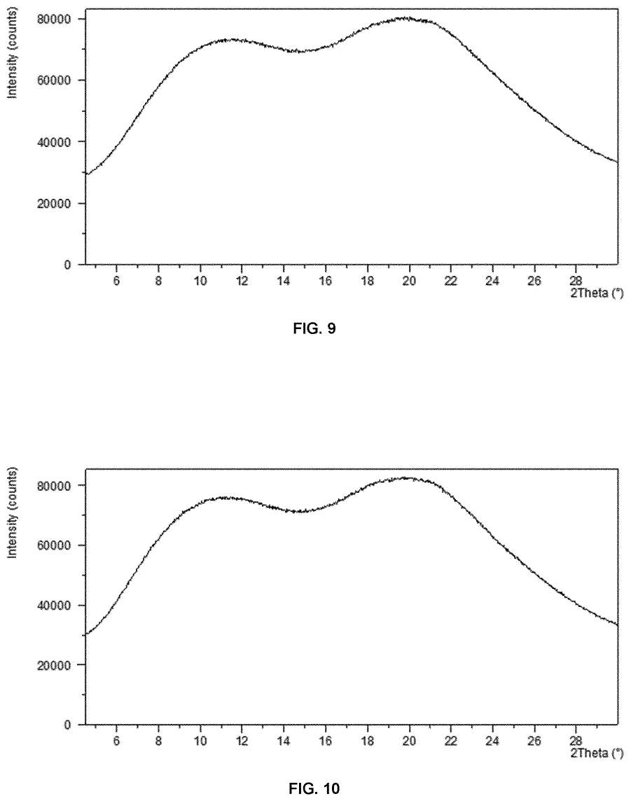

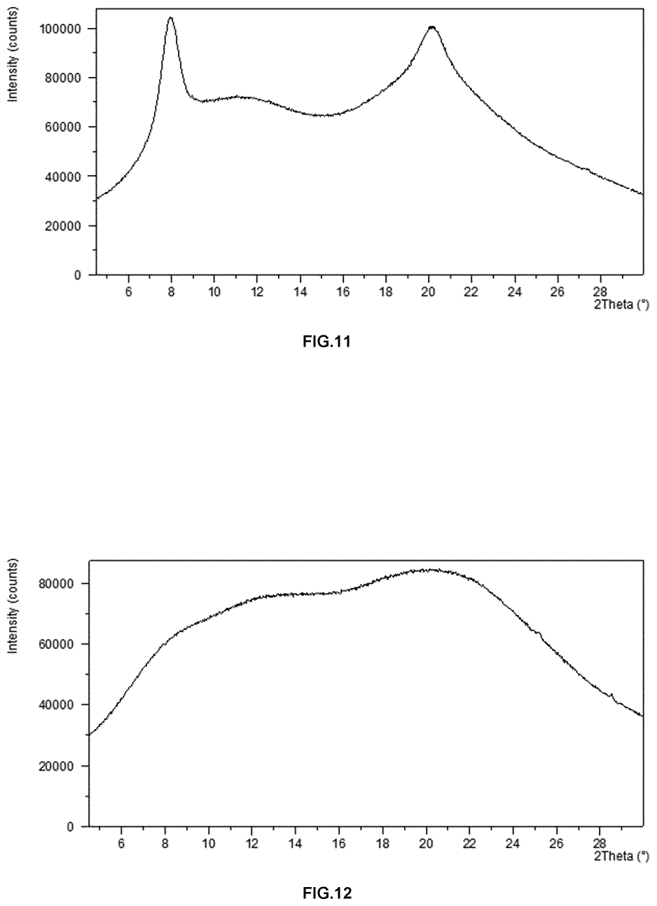

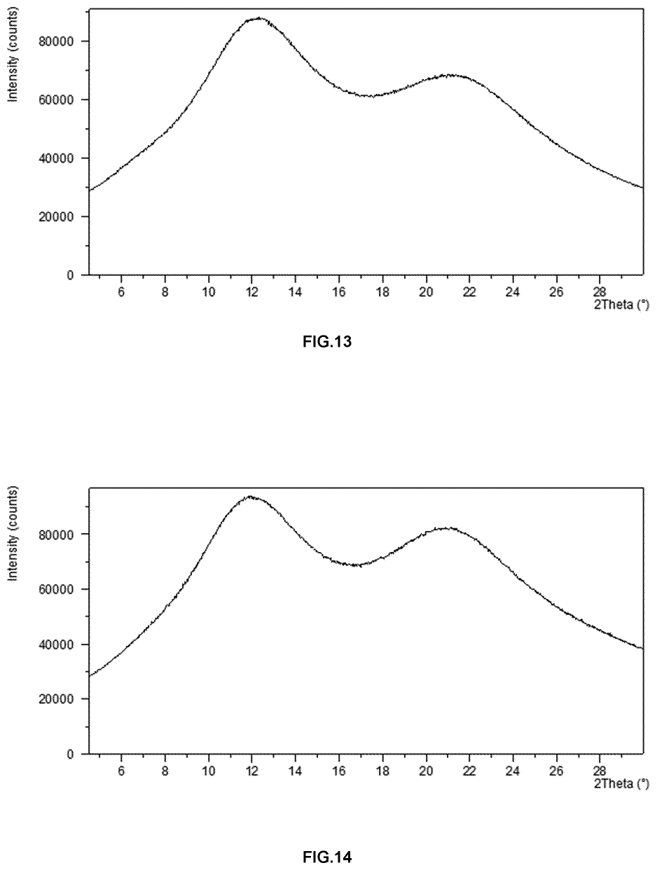

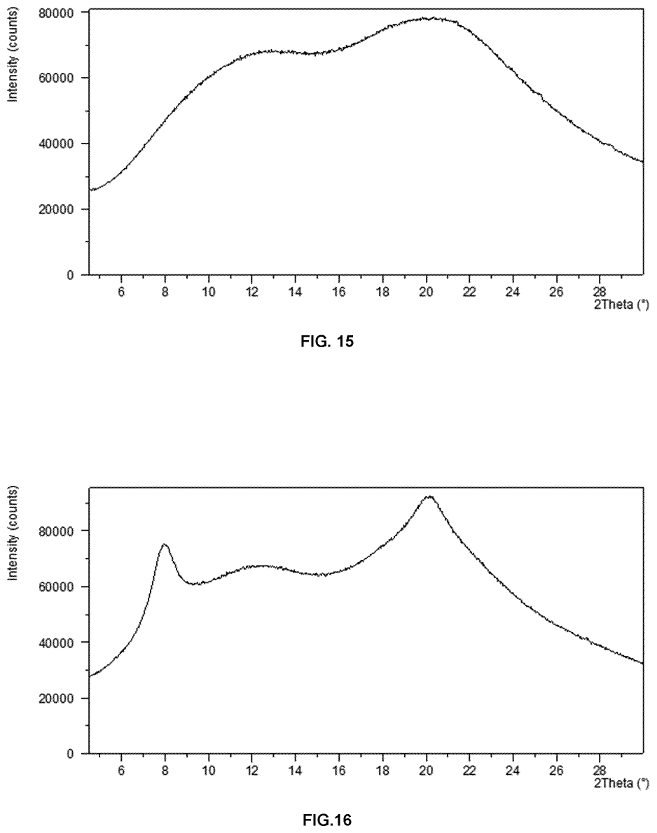

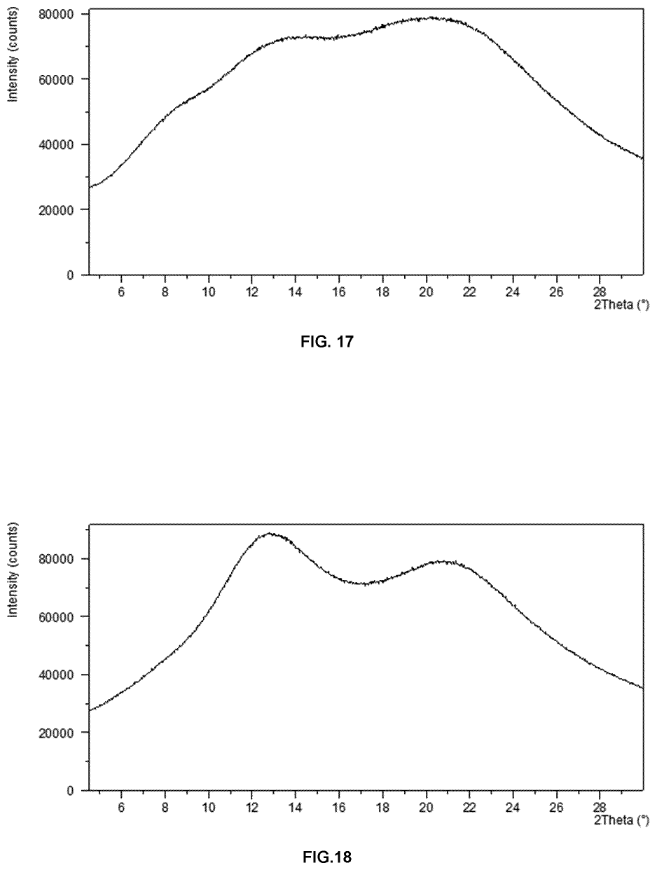

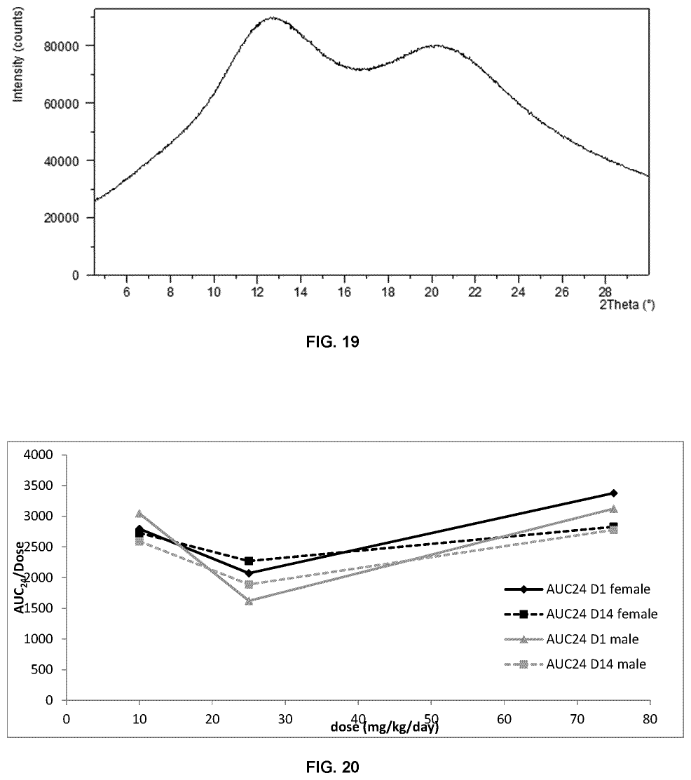

The amorphous solid dispersions of the present invention have been characterized by XRPD as shown in FIGS. 1-4 and 9-19, as described here above and in the Examples.

In addition, the glass transition temperature (Tg) of the amorphous solid dispersions according to the present invention has been measured by Modulated Differential Scanning calorimetry according to methods conventional to the skilled person in the art and as further described in Table 3 of the examples. The Tg is considered to be the temperature where an amorphous solid undergoes an apparent transition from a glass solid state to a supercooled liquid upon heating (see A. Newman and G. Zografi in AAPS PharmSciTech (2020) 21:26). The Tg provides an indication of the miscibility of amorphous compound of formula (I) with the polymer matrix. If a single Tg or a narrow region of Tg is measured, this indicates that the amorphous solid dispersion is homogeneous. This state is also referred to as a glass solution. In addition, the higher the Tg is, the likelihood that the amorphous solid dispersion will have reduced molecular mobility and therefore will remain homogenous over time, which is an indication of its stability.

Amorphous solid dispersions according to the present invention have generally a Tg greater than about 80° C., more generally greater than 100° C., suitably greater than about 105° C., ideally greater than about 110° C., appositely greater than about 115° C., particularly greater than about 120° C.

Amorphous solid dispersions according to the present invention have generally a region of Tg measured, as explained hereabove, which is lower than or equal to about 5° C.

The amorphous solid dispersions according to the present invention are therefore miscible and stable.

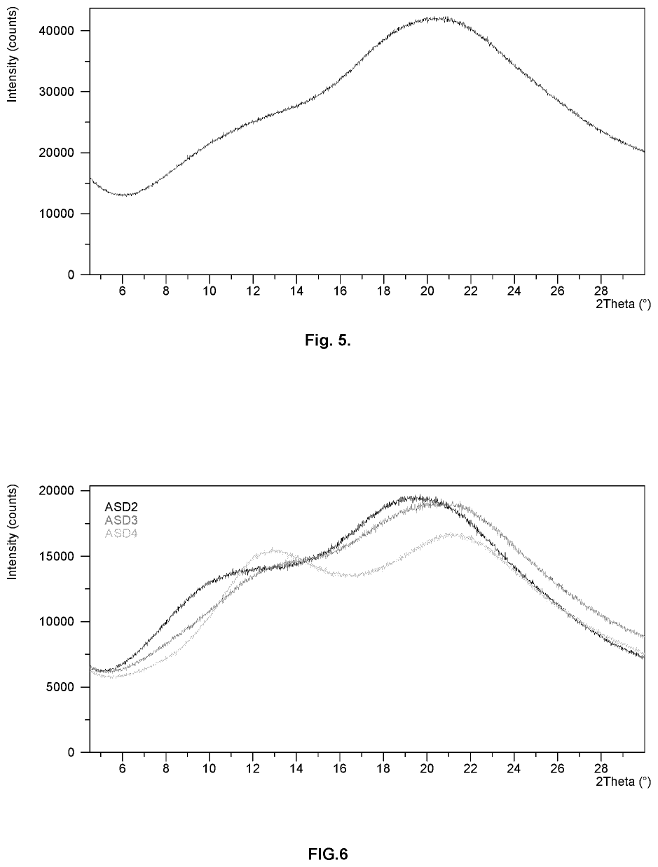

In addition, the stability of some of the amorphous solid dispersions according to the present invention has been tested at room temperature over time as shown in FIGS. 5 and 6 and as further detailed in the Examples. These figures show that these amorphous solid dispersions are stable for at least 10 months at room temperature.

The amorphous solid dispersions of compound of formula (I) according to the present invention are significantly more soluble than the monohydrated crystalline form of compound of formula (I), herein referred to as compound of formula (Ia). Such improved solubility is particularly advantageous when a pharmaceutical composition needs to be prepared, in particular for oral administration, because a higher bioavailabilivity may be achieved. This may also allow reduction of the dose and hence the tablet size to be used when a solid formulation is desired.

Table 4 of the Examples shows comparative solubility data between ASD1-ASD4 and compound of formula (Ia) in different media, showing a minimum 30-fold increase, and up to more than a 100-fold increase, in solubility for the amorphous solid dispersions.

The amorphous solid dispersions according to the present invention may be additionally combined with pharmaceutically acceptable excipients such as diluents, binders, disintegretants, lubricants, glidants or carrier to form a suitable pharmaceutical composition.

Pharmaceutical compositions comprising amorphous solid dispersions according to the present invention may, for example, be administered orally, parenterally, i.e., intravenously, intramuscularly or subcutaneously, intrathecally, by inhalation or intranasally.

Suitable diluents and carriers may take a wide variety of forms depending on the desired route of administration, e.g., oral, rectal, parenteral or intranasal.

Pharmaceutical compositions suitable for oral administration can be solids or liquids and can, for example, be in the form of tablets, pills, dragees, gelatin capsules, solutions, syrups, chewing gums and the like.

Pharmaceutical compositions according to the present invention are generally prepared, according to conventional pharmaceutical compounding techniques known to the skilled practitioner, by mixing the amorphous solid dispersion with inert diluent(s) or a non-toxic pharmaceutically acceptable carrier(s) such as starch or lactose or mannitol or dibasic calcium phosphate Aditionnally, these pharmaceutical compositions can also contain a binder such as microcrystalline cellulose, gum tragacanth or gelatine, a disintegrant such as croscarmellose sodium or crospovidone alginic acid, a lubricant such as magnesium stearate, a glidant such as colloidal silicon dioxide, a sweetener such as sucrose or saccharin, or colouring agents or a flavouring agent such as peppermint or methyl salicylate and coating agents such as Opadry® (I, II, AMB II, QX or EZ).

In a particular embodiment, pharmaceutical compositions according to the present invention are prepared by mixing any one of the amorphous solid dispersions according to the present invention with excipients as further detailed in the process steps described in Example 7.1.

The amount of amorphous solid dispersion in the pharmaceutical compositions can fall within a wide range of concentrations and depends on a variety of factors such as the patient's sex, age, weight and medical condition, as well as on the method of administration. Thus, the quantity of amorphous solid dispersions for oral administration is generally comprised between about 0.5% by weight and about 85% by weight with respect to the total weight of the composition, suitably between about 20% and about 60% by weight with respect to the total weight of the composition.

In a particular embodiment, the present invention relates to a solid pharmaceutical composition which comprises about 20% to about 60% of amorphous solid dispersion per weight compared to the total weight of the uncoated tablet, in association with any of the above-mentioned excipients.

In particular, the present invention relates to a tablet composition which comprises:
Between about 20% and about 60% per weight of amorphous solid dispersion;
Between about 10% and about 50% per weight of lactose monohydrate;
Between about 10% and about 50% per weight of microcrystalline cellulose;
Between about 1% and about 5% per weight of croscarmellose sodium;
Between about 0.1% and about 2% per weight of colloidal anhydrous silica; and
Between about 0.1% and about 5% per weight of magnesium stearate;
Compared to the total weight of the uncoated tablet.

These excipients are generally mixed with amorphous solid dispersions through one or more blending phase and, optionally, a dilution phase as further described in Example 7.1.

In one embodiment the pharmaceutical composition comprises about 25% of amorphous solid dispersion per weight. In another embodiment, the pharmaceutical composition comprises about 50% of amorphous solid dispersion per weight.

In a first embodiment, the pharmaceutical composition comprises about 47.15% per weight of lactose monohydrate. In a second embodiment, the pharmaceutical composition comprises about 27.5% per weight of lactose monohydrate.

In a first embodiment, the pharmaceutical composition comprises about 25.95% per weight of microcrystalline cellulose. In a second embodiment, the pharmaceutical composition comprises about 18.7% per weight of microcrystalline cellulose.

In a first embodiment, the pharmaceutical composition comprises about 1.35% per weight of croscarmellose sodium. In a second embodiment, the pharmaceutical composition comprises about 2.7% per weight of croscarmellose sodium.

In a first embodiment, the pharmaceutical composition comprises about 0.25% per weight of colloidal anhydrous silica. In a second embodiment, the pharmaceutical composition comprises about 0.50% per weight of colloidal anhydrous silica.

In a first embodiment, the pharmaceutical composition comprises about 0.30% per weight of magnesium stearate. In a second embodiment, the pharmaceutical composition comprises about 0.60% per weight of magnesium stearate.

In a particular embodiment, the amorphous solid dispersion is ASD1.

The invention also contemplates compositions which can release the active substance in a controlled manner. Pharmaceutical compositions which can be used for parenteral administration are in conventional form such as aqueous or oily solutions or suspensions generally contained in ampoules, disposable syringes, glass or plastics vials or infusion containers.

In addition to the amorphous solid dispersion, these solutions or suspensions can optionally also contain a sterile diluent such as water for injection, a physiological saline solution, oils, polyethylene glycols, glycerine, propylene glycol or other synthetic solvents, antibacterial agents such as benzyl alcohol, antioxidants such as ascorbic acid or sodium bisulphite, antifoam agents, chelating agents such as ethylene diamine-tetra-acetic acid, buffers such as acetates, citrates or phosphates and agents for adjusting the osmolarity, such as sodium chloride or dextrose, and viscosifying agents such as Hydroxypropylcellulose (HPC-SSL), hypromellose derivatives (HPMC) and finally stabilizing agents such as PVPVA, PVP, and polyvinyl alcohol (PVA).

These pharmaceutical forms are prepared using methods which are routinely used by pharmacists.

International patent application n° PCT/EP2020/068183, published as WO2021/001288, describes that compound of formula (I) may be useful for the treatment of diseases and/or disorders in which D1 receptors play a role, and in particular cognitive and negative symptoms in schizophrenia, cognitive impairment related to neuroleptic therapy, Mild Cognitive impairment (MCI), impulsivity, Attention-Defficit Hyperactivity Disorder (ADHD), Parkinson's disease and other movement disorders, dystonia, Parkinson's dementia, Huntington's disease, dementia with Lewy Body, Alzheimer's disease drug addiction, sleep disorders, apathy, traumatic spinal cord injury or neuropathic pain.

Thus, in a further aspect, the present invention provides for amorphous solid dispersions as described herein, or pharmaceutical composition thereof, for use in the treatment and/or prevention of cognitive and negative symptoms in schizophrenia, cognitive impairment related to neuroleptic therapy, Mild Cognitive impairment (MCI), impulsivity, Attention-Defficit Hyperactivity Disorder (ADHD), Parkinson's disease and other movement disorders, dystonia, Parkinson's dementia, Huntington's disease, dementia with Lewy Body, Alzheimer's disease drug addiction, sleep disorders, apathy, traumatic spinal cord injury or neuropathic pain.

In a particular aspect, the present invention provides for amorphous solid dispersions as defined above, or pharmaceutical compositions thereof, for use in the treatment of Parkinson's disease and other movement disorders, Alzheimer's disease, or cognitive and negative symptoms in schizophrenia.

The present invention also provides for the use of amorphous solid dispersions as described herein, or pharmaceutical composition thereof, for the manufacture of a medicament for use in the treatment and/or prevention of cognitive and negative symptoms in schizophrenia, cognitive impairment related to neuroleptic therapy, Mild Cognitive impairment (MCI), impulsivity, Attention-Defficit Hyperactivity Disorder (ADHD), Parkinson's disease and other movement disorders, dystonia, Parkinson's dementia, Huntington's disease, dementia with Lewy Body, Alzheimer's disease drug addiction, sleep disorders, apathy, traumatic spinal cord injury or neuropathic pain.

In a particular aspect, the present invention provides for the use of amorphous solid dispersions as defined above, or pharmaceutical compositions thereof, for the manufacture of a medicament for the treatment of Parkinson's disease and other movement disorders, Alzheimer's disease, or cognitive and negative symptoms in schizophrenia.

The present invention also provides for a method of treatment of and/or prevention of cognitive and negative symptoms in schizophrenia, cognitive impairment related to neuroleptic therapy, Mild Cognitive impairment (MCI), impulsivity, Attention-Defficit Hyperactivity Disorder (ADHD), Parkinson's disease and other movement disorders, dystonia, Parkinson's dementia, Huntington's disease, dementia with Lewy Body, Alzheimer's disease drug addiction, sleep disorders, apathy, traumatic spinal cord injury or neuropathic pain which comprises administering to a patient in need of such treatment an effective amount of amorphous solid dispersion as described herein, or pharmaceutical composition thereof.

In a particular aspect, the present invention provides for a method of treatment of and/or prevention of of Parkinson's disease and other movement disorders, Alzheimer's disease, or cognitive and negative symptoms in schizophrenia which comprises administering to a patient in need of such treatment an effective amount of amorphous solid dispersion as described herein, or pharmaceutical composition thereof.

### Examples

### Abbreviations/Recurrent Reagents

ACN: Acetonitrile
Brine: Saturated aqueous sodium chloride solution
nBu: n-butyl
tBu: tert-butyl
Cellulose, microcrystalline: Avicel PH-105 & PH-200 (commercial name)
Croscarmellose sodium: Ac-Di-Sol (commercial name)
cAMP: cyclic adenosinemonophosphate
DCM: Dichloromethane
DMAP: 4-Dimethylaminopyridine
DMF: N,N-Dimethylformamide
DMSO: Dimethylsulfoxide.
mDSC: modulated Differential Scanning calorimetry.
ES⁺: Electrospray Positive Ionisation
Et: Ethyl
EtOH: Ethanol
Et₂O: Diethyl ether
EtOAc: Ethyl acetate
h: Hour
HPLC: High Performance Liquid Chromatography
HTRF: homogenous time-resolved fluorescence
HPCMAS-L: Hydroxypropyl methylcellulose acetate succinate L grade.
HPMCAS-M: Hydroxypropyl methylcellulose acetate succinate M grade
HPMC E3LV: Hypromellose E3LV grade.
HPMC 15LV: Hypromellose 15LV grade (commercial name Affinisol).
HPMC 100LV: Hypromellose 100LV grade (commercial name Affinisol).
HPMCP HP-55: Hypromellose phthalate HP55 gradeLactose monohydrate: FlowLac 90 (commercial name)
LCMS: Liquid Chromatography Mass Spectrometry
Magnesium stearate: HyQual 2257 (commercial name)
MeOH: Methanol
min.: minutes
NCS: N-Chlorosuccinimide
NMR: Nuclear magnetic resonance
iPrOH: isopropanol
PVPVA 64: copolymer N-vinyl-2-pyrrolidone/vinyl acetate.
PVP 17PF: polyvinylpyrrolidone 17PF grade.
rt: room temperature
SFC: Supercritical Fluid Chromatography
Silica, colloidal anhydrous: Cab-O-Sil M-5P (commercial name)
TEA: Triethylamine
THF: Tetrahydrofuran
TLC: Thin Layer Chromatography
Tg: glass transition temperature.
XRPD: X-Ray Powder Diffraction.
IUPAC names have been determined using Biovia Draw 16.1.

### 1. Analytical Methods

All reactions involving air or moisture-sensitive reagents were performed under a nitrogen or argon atmosphere using dried solvents and glassware. Commercial solvents and reagents were generally used without further purification, including anhydrous solvents when appropriate (generally Sure-Seal™ products from Aldrich Chemical Company or AcroSeal™ from ACROS Organics). In general reactions were followed by thin layer chromatography, HPLC or mass spectrometry analyses according to conventional methods known to the person skilled in the art.

Crude materials could be purified by normal phase chromatography, (acidic or basic) reverse phase chromatography, chiral separation or recrystallization.

Products were generally dried under vacuum before final analyses and submission to biological testing.

All NMR spectra were obtained at 250 MHz, 300 MHz, 400 MHz or 500 MHz.

The compounds were studied in DMSO-d₆, CDCl₃ or MeOH-d 4 solution at a probe temperature of 300 K and at a concentration of 10 mg/mL. The instrument is locked on the deuterium signal of DMSO-d₆, CDCl₃ or CD₃OD. Chemical shifts are given in ppm downfield from TMS (tetramethylsilane) taken as internal standard.

### 2. Preparation of Monohydrated Crystalline Form of 2-(3,5-dichloro-1-methyl-indazol-4-yl)-1-[(1S,3R)-3-(hydroxymethyl)-5-(1-hydroxy-1-methyl-ethyl)-1-methyl-3,4-dihydro-1H-isoquinolin-2-yl]ethanone (Ia)

Compound of formula (Ia) was prepared by applying the same synthetic method described in Example 2 of co-pending international patent application WO 2021/001288, herein incorporated by reference.

The following recristallization protocol was also applied as an alternative to the recristallization protocol disclosed in section 2.8:

A recristallization is carried out on 5.00 g Crude material is solubilized in 240 ml of dimethylsulfoxide. The solution is heated to 40° C. then filtered on a P3 sintered glass. The reactor and filter are rinsed with 35 ml dimethylsulfoxide. The filtrate is transferred to a clean reactor and heated to 85° C. 110 ml of water are dosed slowly over 30 minutes. 250 mg of compound (Ia) (0.5% w/w, monohydrate form) are then added to the reaction mixture. The mixture is stirred for 2h30 at 85° C. while crystalline material comes out of solution before being cooled down slowly to 20° C. over 12 hours. The suspension is filtered and the filtercake is rinsed successively with several portions of water and then with 150 ml of ethyl acetate. The filtercake is dried under vacuum at 50-60° C. Compound (Ia) is obtained as 46.9 g of an off-white powder. Yield=94%

¹H NMR (400 MHz, DMSO-d₆) δ 7.65 (dd, J=9.0, 2.2 Hz, 1H), 7.52 (dd, J=9.0, 2.1 Hz, 1H), 7.37 (ddd, J=19.6, 7.6, 1.7 Hz, 1H), 7.25-7.03 (m, 2H), 5.30 (q, J=6.5 Hz, 0.3H), 5.16-4.99 (m, 1.7H), 4.99-4.84 (m, 0.7H), 4.63-4.30 (m, 3.3H), 4.17-3.93 (m, 4H), 3.28 (dt, J=10.5, 5.1 Hz, 1.3H), 3.10-2.85 (m, 1.7H), 1.56 (dd, J=13.2, 6.9 Hz, 6.7H), 1.24 (d, J=6.5 Hz, 2.3H).

### 3. Preparation and Characterization of Amorphous Solid Dispersions of 2-(3,5-dichloro-1-methyl-indazol-4-yl)-1-[(1S,3R)-3-(hydroxymethyl)-5-(1-hydroxy-1-methyl-ethyl)-1-methyl-3,4-dihydro-1H-isoquinolin-2-yl]ethanone

3.1. Spray Drying Protocol

Compound (Ia) and carrier are dissolved into organic solvent(s) and spray dried to afford different solid dispersions as set out in paragraph 3.1.a. and Table 1 below. Polymer matrix used is generally commercially available and may be obtained in different grade quality.

Different types of spray drying equipments can be used. The spray-drying equipment used in the present protocol is ProCept 4M8-TriX (ProCept, Belgium).

3.1.a. Synthesis of Amorphous Solid Dispersion 1 (ASD1)

About 40 g of compound of formula (Ia) and about 60 g of commercially available HPCMAS-L, corresponding to a weight ratio of about 40/60 weight %, are fully dissolved into a mixture of dichloromethane/methanol 76/24 weight % to reach a total solid content of around 5% (w/w) in solution. Then, the feed solution is pumped to the bi-fluid nozzle at a rate of 18 g/min under a pressure of 1.5 bar and atomized into fine droplets. The solvent is evaporated through a co-current drying airflow set up at an inlet temperature of 65° C. Atomization and drying parameters are adjusted to achieve an outlet temperature of 40-45° C. Once evaporated, the dried particles are then collected through a cyclone. The collected wet material is stored in a vacuum oven for an additional 12 hours at a temperature of 25° C. to afford about 85 g of the desired solid dispersion of amorphous 2-(3,5-dichloro-1-methyl-indazol-4-yl)-1-[(1S,3R)-3-(hydroxymethyl)-5-(1-hydroxy-1-methyl-ethyl)-1-methyl-3,4-dihydro-1H-isoquinolin-2-yl]ethanone, referred to as ASD1. Yield=about 85% (Material collected after secondary drying/Material dissolved in feed solution) %

Other amorphous solid dispersions may be prepared in a similar fashion, using the quantities of compound of formula (Ia) and polymer matrix as summarized in Table 1 hereunder.

TABLE 1
composition of ASD 2-4

| | Weight % of | | Weight % of |
|---|---|---|---|
| ASD | compound (Ia) | Polymer matrix | polymer matrix |
| ASD2 | ~30% | HPCMAS-L | ~70% |
| ASD3 | ~60% | HPCMAS-L | ~40% |
| ASD4 | ~40% | PVPVA | ~60% |

3.2. Hot Melt Extrusion Protocol

About 10 g of a powder mixture of compound (Ia) and polymer matrix in the weight proportions mentioned in Table 2 was prepared. For example, when 40 weight % of (Ia) is indicated this represents about 4.0 g of compound (Ia) and about 6.0 g of polymer matrix.

Polymer matrix used are generally commercially available and may be obtained in different grade quality. Compound (Ia) and polymer matrix are blended with 3-Dimensional shaker system TURBULA® (WAB) for 5 minutes. The pre-blend was then de-lumped with #25 mesh (˜700 μm) screen and blended for an additional 5 minutes duration with TURBULA® system. The pre-mixed powders were then manually fed into the bench-scale hot melt extruder (Thermo Scientific HAAKE™ MiniCTW Micro-Conical Twin Screw Compounder, ThermoFisher) operating in a counter-rotating screw configuration. The barrel temperature was fixed at 160° C. and the screw speed was defined at 200 rpm. A force feeder was used to fill the barrel with an auger speed fixed at 500 rpm. Extrudates were collected after cooling to ambient temperature. After a single pass extrusion in the barrel, visual observation was conducted on each extrudate. If extrudate does not appear transparent, material was recycled in the barrel for additional 2 min and extruded. Following extrusion, the material was milled for 1-2 minutes with small burr-type mill system while passing through #60 mesh (˜250 μm) screen. Milled extrudate was sieved through 250 μm sieve and the different fractions were stored separately.

TABLE 2

| | | | Weight of | |
|---|---|---|---|---|
| | | | particles of | Yield particles |
| | | Polymer | size ≤250 | of size ≤250 |
| ASD | Weight % (Ia) | matrix | micron (g) | micron (%) |
| ASD5 | 40% | HPMCAS-L | 2.952 | 44 |
| ASD6 | 40% | HPMCAS-M | 5.036 | 56 |
| ASD7 | 40% | HPMC E3LV | 4.417 | 52 |
| ASD8 | 40% | HPMCP HP-55 | 5.295 | 59 |
| ASD9 | 40% | PVPVA 64 | 5.529 | 57 |
| ASD10 | 40% | PVP 17PF | 4.826 | 50 |
| ASD11 | 50% | HPMCAS-L | 2.939 | 41 |
| ASD12 | 50% | HPMC E3LV | 5.179 | 65 |
| ASD13 | 50% | HPMCP HP-55 | 5.129 | 64 |
| ASD14 | 50% | PVPVA 64 | 5.166 | 60 |
| ASD15 | 50% | PVP-17PF | 5.048 | 57 |
| ASD16 | 40% | HPMC 15LV | / | / |
| ASD17 | 40% | HPMC 100LV | / | / |

3.3. X-Ray Powder Diffraction (XRPD) of ASD1-ASD17

The amorphous solid dispersions ASD1-ASD17, obtained respectively by spray drying and hot melt extrusion as described herein, have been characterized by XRPD according to the following general protocol.

X-Ray powder patterns for ASD1-ASD15 were obtained with a PANalytical Empyrean Serie 2 X-ray powder diffractometer using a Cu Kα radiation, equipped with the Bragg-BrentanoHD optical module for the incident beam path, in reflection geometry, and a PIXel 3D detector. The Data Collector software was used to record the data. The tube voltage and amperage were set to 45 kV and 40 mA, respectively, during measurement. Samples, placed either in a flat zero background, a zero background cup or a backloading sample holders, were analyzed between 4,5 and 30° 2-theta at a scan speed between 0.2 and 2.1°/min. Data was processed using Data Viewer or HighScore Plus. X-Ray powder patterns for ASD16 and ASD17 were obtained with a Rigaku Miniflex 6G X-ray diffractometer using a Cu Kα radiation in reflection geometry. The tube voltage and amperage were set to 40 kV and 15 mA, respectively, during measurements. Samples, placed either in zero background cup or in zero background low volume cup, were analyzed between 3 and 30° 2-theta at a scan speed of 0.9°/min. Data was processed using Data Viewer or HighScore Plus.

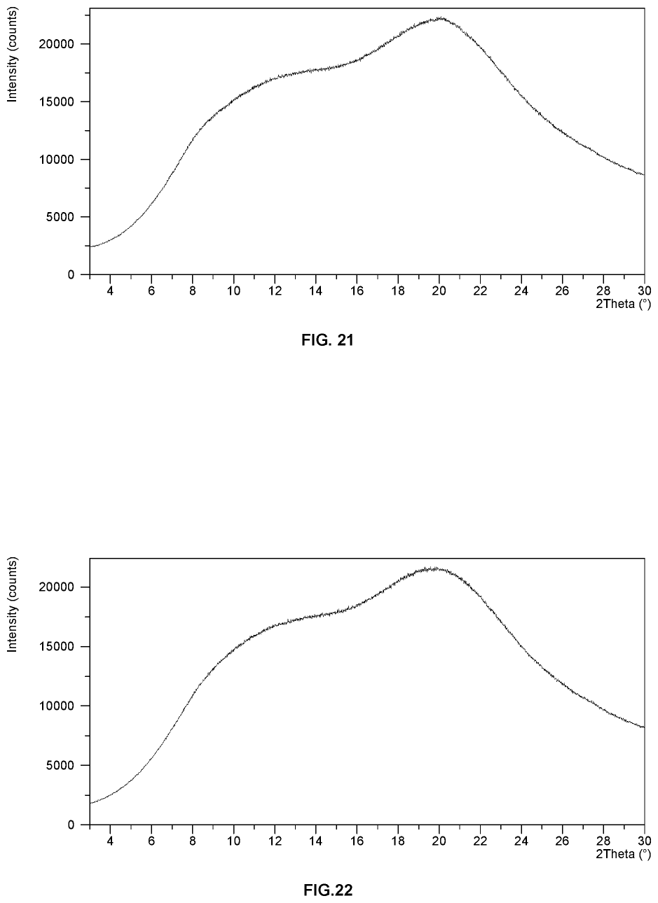

FIGS. 1-4 and 9-19, 21 and 22 show the XPRD pattern of ASD1-ASD17 which display a classical amorphous solid-state halo. Note that the peaks which appear in the patterns of ASD7 and ASD12 are not due to the presence of a crystalline form of compound of formula (I) but to some impurities coming from the polymer matrix.

### 3.4. Differential Scanning Calorimetry (DSC) of Solid Dispersions of Amorphous 2-(3,5-dichloro-1-methyl-indazol-4-yl)-1-[(1S,3R)-3-(hydroxymethyl)-5-(1-hydroxy-1-methyl-ethyl)-1-methyl-3,4-dihydro-1H-isoquinolin-2-yl]ethenone

The phase behavior and thermal properties of ASD1-15 obtained under paragraphs 3.1. and 3.2 were analyzed by modulated DSC (mDSC) using TA Instruments Q1000 calorimeter (TA Instruments, Leatherhead, UK). The chamber was purged with a 50 mL/min flow rate of dry nitrogen. Indium and sapphire disks were used to calibrate the temperature/enthalpy and heat capacity, respectively. The powder was analyzed in non-hermetic standard aluminium pans (TA Instruments, Leatherhead, UK). In a typical mDSC analysis, the samples were heated from 0° C. to 250° C. at 2° C./min with a modulation of ±1° C. and for a period of 40-60 seconds. Data was processed using Universal Analysis 2000 software (TA Instruments, Leatherhead, UK). Glass temperature (Tg) was reported as the mid-point of inflection in the step change observed in the reverse heat flow signal while crystallization and melting events were recorded in non-reverse and total heat flows.

Table 3 summarizes the Tg value obtained for ASD1-ASD17

| ASD | Tg (mDSC) |
|---|---|
| ASD1 | 108-112° C. |
| ASD2 | 105-110° C. |
| ASD3 | 110-115° C. |
| ASD4 | 120-125° C. |
| ASD5 | 105-110° C. |
| ASD6 | 105-110° C. |
| ASD7 | 108-112° C. |
| ASD8 | 110-115° C. |
| ASD9 | 115-120° C. |
| ASD10 | 135-140° C. |
| ASD11 | 100-105° C. |
| ASD12 | 105-110° C. |
| ASD13 | 108-112° C. |
| ASD14 | 110-115° C. |
| ASD15 | 130-135° C. |
| ASD16 | 83-87° C. |
| ASD17 | 83-87° C. |

### 3.5. Stability of ASD1, ASD2, ASD3 and ASD4

XRPD of ASD1-ASD4 displayed in FIGS. 1-4 have been taken at t=0.

Additionally, XRPD of ASD1 has been taken after 12 months at 25° C. and 60% relative humidity and resulting pattern is displayed FIG. 5.

XRPD of ASD2, ASD3, and ASD4 were also additionally taken after 10 months at room temperature in the presence of silicagel as dessicant, and the resulting patterns are displayed in FIG. 6.

These studies show that ASD1-ASD4 are all stable for at least 10 months.

### 4. Comparative Solubility of Solid Dispersions of Amorphous 2-(3,5-dichloro-1-methyl-indazol-4-yl)-1-[(1S,3R)-3-(hydroxymethyl)-5-(1-hydroxy-1-methyl-ethyl)-1-methyl-3,4-dihydro-1H-isoquinolin-2-yl]ethanone with compound of formula (Ia)

The respective solubility of compounds of formula (Ia) and ASD1 was determined in different media while using the shake flask method. An excess of solid (equivalent to a concentration of compound of formula (I) of 5 mg/mL) was suspended in 5 mL of buffer/biorelevant media as specifically described in Table 4 and incubated in a sealed glass vial (10 mL) for 24 h at both RT and 37° C. in a climatic chamber equipped with a rotary mixer. The 24h time point was assumed to have reached solubility, at which time the suspension was filtered through a 0.45-μm ultra free filter (Merck Millipore) and the drug content was determined by HPLC. The solubility of compound (Ia) and ASD1 was determined in triplicate (n=3). Where required to prevent drug precipitation, the filtrate was diluted with a suitable organic solvent. Table 4 below shows the solubility of compound (Ia) compared with solubility of ASD1 in different media. These media are respectively a phosphate buffer, FasSGF, FASSIF-V2 and FeSSIF-V2. FasSG is fasted condition gastric fluid. FasSGF is prepared at pH 1.6 and contains 0.08 mM Taurocholate, 0.02 mM phospholipids, 34 mM sodium and 59 mM chloride. FaSSIF-V2 and FeSSIF-V2 are Fasted and Fed State Biorelevant Media, respectively. FaSSIF-V2 is prepared at pH 6.5 and contains 3 mM Taurocholate, 0.2 mM Phospholipids, 106 mM Sodium, 69 mM Chloride and 19 mM Maleic acid. FeSSIF-V2 is prepared at pH 5.8 and contains 10 mM Taurocholate, 2 mM Phospholipids, 0.8 mM Oleate, 5 mM Glycerol monoleate, 218 mM Sodium, 125 mM Chloride and 55 mM Maleic acid.

TABLE 4

| | | Solubility Measurements | | | |
|---|---|---|---|---|---|
| | | (24 h) | | | |
| Media | pH/temperature | Compound (Ia) | | ASD1 | |
| Phosphate buffer | 5.8/RT | <1 | μg/mL | 60 ± 20 | μg/mL |
| (50 mM) | 6.5/RT | <1 | μg/mL | 115 ± 20 | μg/mL |
| FasSGF | 1.6/37° C. | <1 | μg/mL | 30 ± 15 | μg/mL |
| FaSSIF-V2 | 6.5/37° C. | ~1.5 | μg/mL | 215 ± 30 | μg/mL |
| FeSSIF-V2 | 5.8/37° C. | ~4 | μg/mL | 350 ± 20 | μg/mL |

The results obtained hereabove show that a minimum of 30-fold increase, and up to more than a 100-fold increase, in solubility for ASD1 compared to compound of formula (Ia) is obtained.

### 5. Dissolution Profile of ASD1 and Compound of Formula (Ia)

5.1 General Protocol

The dissolution profile was determined in a USP Apparatus 2 type (Distek 2100 C Dissolution Apparatus) at 37° C. The dynamic dissolution test includes first a 30 minutes dissolution in simulated gastric media (0.1N HCl) to achieve a concentration equivalent to a concentration of compound of formula (I) of 1 mg/mL, followed by a 180 minutes dissolution in FaSSIF-V2 to achieve a concentration equivalent to a concentration of compound of formula (I) of 0.5 mg/mL.

5.2. Dissolution of ASD1

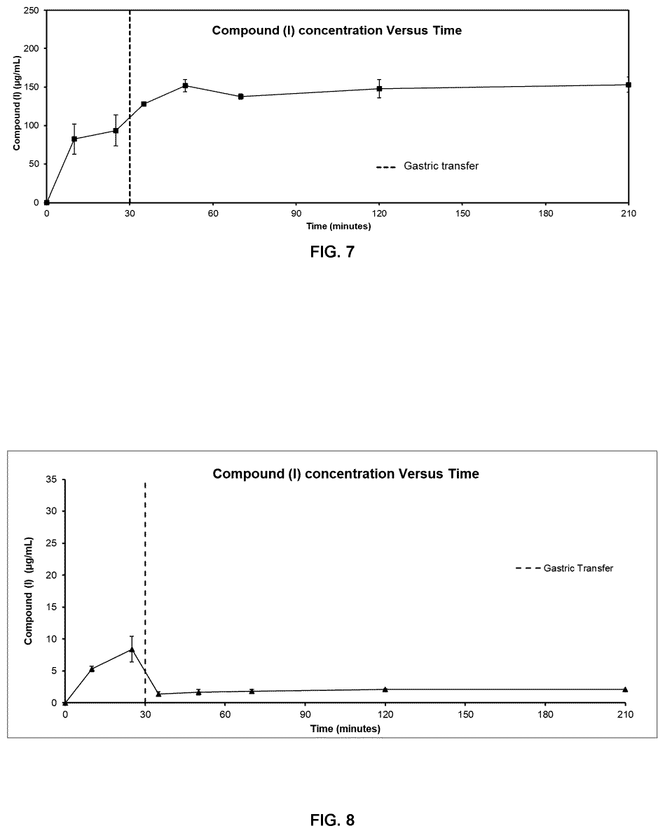

125 mg of ASD1 was weighed and disposed into a 100 mL vessel. Then, 50 mL of simulated gastric media (0.1N HCl) was added to the vessel and the paddle speed was fixed at 100 rpm. After 30 minutes, an equivalent volume (50 mL) of fasted state biorelevant media was added to the vessel to obtain the composition of FaSSIF-V2. Dissolution was carried out in triplicate (n=3). At each timepoint, the suspension was filtered through a 0.45-μm ultra free filter (Merck Millipore) and the content of compound of formula (I) was determined by HPLC. The filtrate was then diluted with a suitable organic solvent. The dissolution profile of ASD1 is represented in FIG. 7.

Dissolution Profile of Compound (Ia)

FIG. 8 shows the dissolution profile of compound (Ia) performed in analogous conditions to the ones described herabove.

From a comparison between FIGS. 7 and 8, it can be deduced that ASD1 dissolves rapidly in gastric media compared to compound of formula (Ia) and remains very soluble for a a few hours, whereas the solubility of compound (Ia) drops in gastric media to very low levels during the same period of time. Overall, ASD1 was found to generate and maintain supersaturation during the duration of the experiment.

This confirms that amorphous solid dispersions according to the present invention, in particular ASD1, have an improved solubility profile over compound of formula (Ia) and therefore advantageous properties.

### 6. In Vivo Bioavailability of a Liquid Suspension of ASD1

6.1. Liquid Suspension of ASD1

The formulation vehicle used in the following suspension is a mixture of 1% (w/v) Hydroxypropyl cellulose grade SSL, 10% (w/v) PVPVA, 0.1% (w/v) Antifoam 1510 US in 50 mM Citrate buffer pH 3.0 in water.

First, a 50 mM Citrate buffer pH 3.0 in water was prepared. Secondly, Hydroxypropyl cellulose grade SSL, PVPVA and Antifoam 1510 US were successively dissolved in the freshly prepared citrate buffer and stirred (magnetic stirring) for 120 minutes.

15.0 g of ASD1 was weighed and disposed into the container. 88.8 g of the prepared vehicle was added to ASD1 while mixing manually by means of a glass stick or inox spatula. 88.8 g additional vehicle was then added and the suspension was then stirred for an additional 30 minutes at 250 rpm. Unless constant stirring is maintained, the suspension is stirred again using magnetic bar/stirring for 15 minutes before animals are dosed and throughout administration.

6.2. Administration and Bioavailability Measurements.

Four groups of 2 male and 2 female dogs were treated with ASD1 in suspension prepared according to Example 6.1. at respective doses of 10, 25 and 75 mg/kg/day for 14 consecutive days.

Plasma samples were collected on Day 1 and 14, at different time points after dosing: 1h, 2h, 4h, 7h, 12h and 24 hours after dosing.

Plasma concentrations of compound (I) were quantified by LC/MS (liquid chromatography/mass spectrometry).

The area under the curve (AUC₂₄) were calculated at day 1 and day 14 between 0 and 24h using the log-lin interpolation rule. This AUC₂₄ is divided by the dose administered and is plotted as a function of the dose, as displayed in FIG. 20.

FIG. 20 shows that the AUC₂₄ of compound of formula (I) increased proportionally with the dose administered, thus indicating that ASD1 in suspension maintained the same level of bioavailability when increasing the dose from 10 to 75 mg/kg.

### 7. Tablets Containing ASDs

7.1. Preparation of Tablets Containing Amorphous Solid Dispersions and Tablet Composition

Amorphous solid dispersions obtained according to Example 3 are formulated into tablets according to the general process steps described here below, following methods which are generally known to the person skilled in the art:
1) The amorphous solid dispersion is blended with suitable excipients such as microcrystalline cellulose, lactose monohydrate, croscarmellose sodium, anhydrous colloidal silica, and magnesium stearate;
2) The blend obtained in step 1, herein referred to as Blend #1, is compacted by dry granulation and then milled;
3) The granules obtained under step 2 are further blended with suitable excipients such as microcrystalline cellulose, croscarmellose sodium and magnesium stearate.
4) The blend obtained as a result of step 3, herein after referred to as Blend #2, is compressed, after an optional dilution with microcrystalline cellulose and lactose monohydrate for certain dosage strengths, to afford the uncoated tablet;
5) the uncoated tablet is spray-coated with a suitable coating agent, such as Opadry® (I, II, AMB II, QX or EZ).

Examples of tablets obtained by applying above mentioned process steps to ASD1 are composed as follows:

| Tablet | A (mg) | B (mg) | C (mg) | D (mg) |
|---|---|---|---|---|
| Total uncoated tablet | 100 | 100 | 250 | 500 |
| weight | | | | |
| Step 1-Blend #1 | | | | |
| ASD 1 (mg) | 25.00 | 50 | 125 | 250 |
| Lactose monohydrate | 13.75 | 27.50 | 68.75 | 137.50 |
| Cellulose, | 6.85 | 13.70 | 34.25 | 68.50 |
| microcrystalline | | | | |
| Croscarmellose sodium | 1.00 | 2.00 | 5.00 | 10.00 |
| Silica, colloidal | 0.25 | 0.50 | 1.25 | 2.50 |
| anhydrous | | | | |
| Magnesium stearate | 0.15 | 0.30 | 0.75 | 1.50 |
| Step 3-Blend #2 | | | | |
| Cellulose, | 2.50 | 5.00 | 12.50 | 25.00 |
| microcrystalline | | | | |
| Croscarmellose sodium | 0.35 | 0.70 | 1.75 | 3.50 |
| Magnesium stearate | 0.15 | 0.30 | 0.75 | 1.50 |
| Step 4 | | | | |
| Lactose monohydrate | 33.40 | / | / | / |
| Cellulose, | 16.60 | / | / | / |
| microcrystalline | | | | |

Coated tablets A, B, C and D contain additionally and respectively about 4, 4, 10 and 20 mg of Opadry AMB II 88A180040 white.

7.2. Dissolution Profile of Tablets a, B, C and D

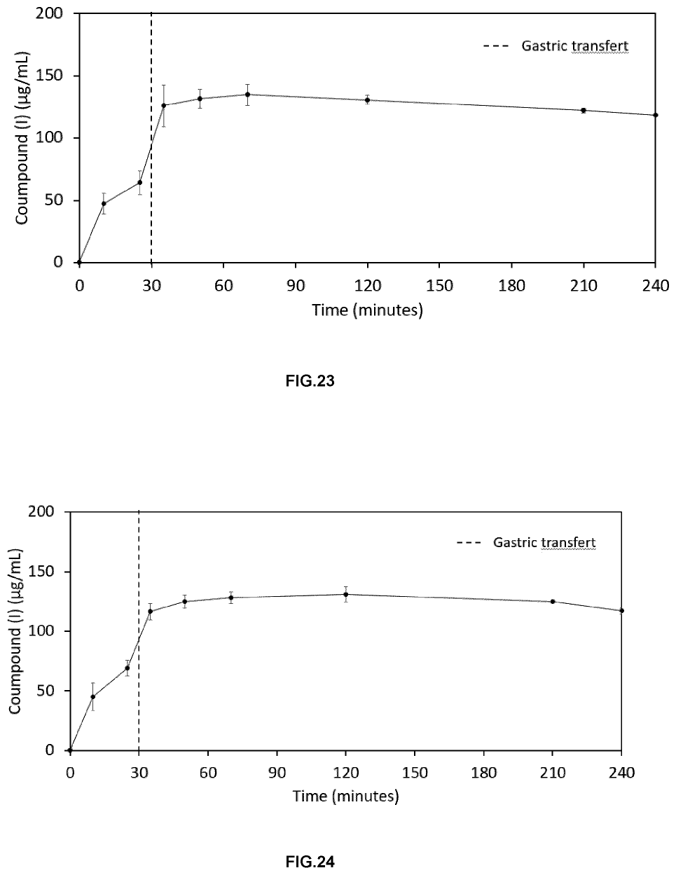

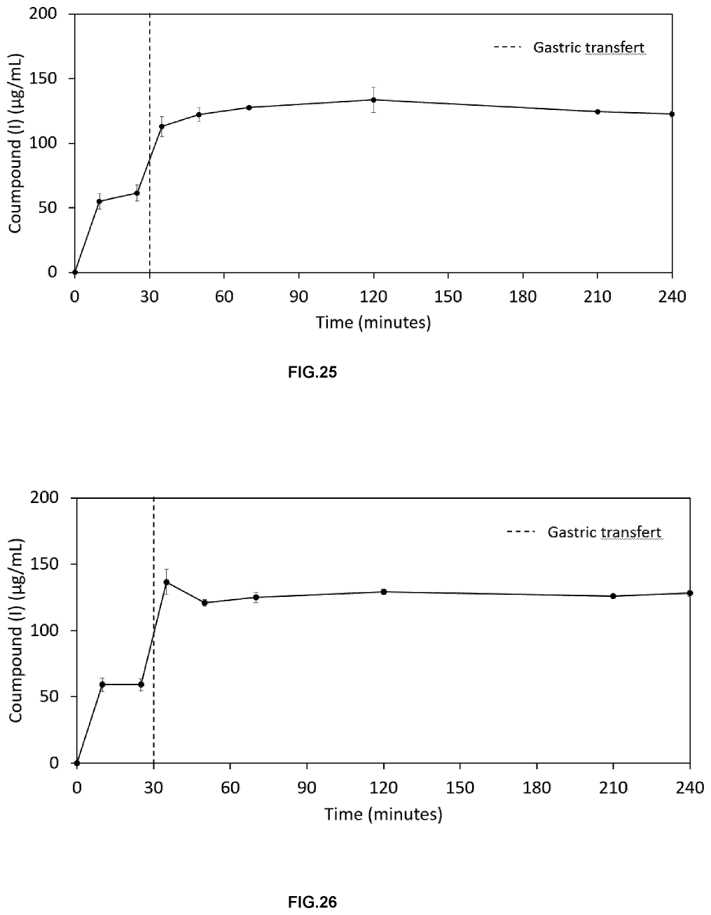

Dissolution profiles of the coated Tablets A, B, C & D were measured according to the protocol described in Example 5 and are shown respectively FIGS. 23, 24, 25 and 26. Note that the number of tablets or volume of dissolution media can be adapted to reach the desired target concentration.

Results obtained with Tablets A, B, C and D show that amorphous solid dispersions and their corresponding solid formulation are advantageous in terms of solubility and dissolution rate compared to the monohydrated crystalline compound of formula (Ia).

7.3. Stability of Tablets a, B, C and D

Coated Tablets A, B, and D packaged into High Density PolyEthylene bottles with 2 g silica gel desiccant housed in the twist off cap were found to be stable after 12 months of storage at 25° C. and 60% relative humidity.

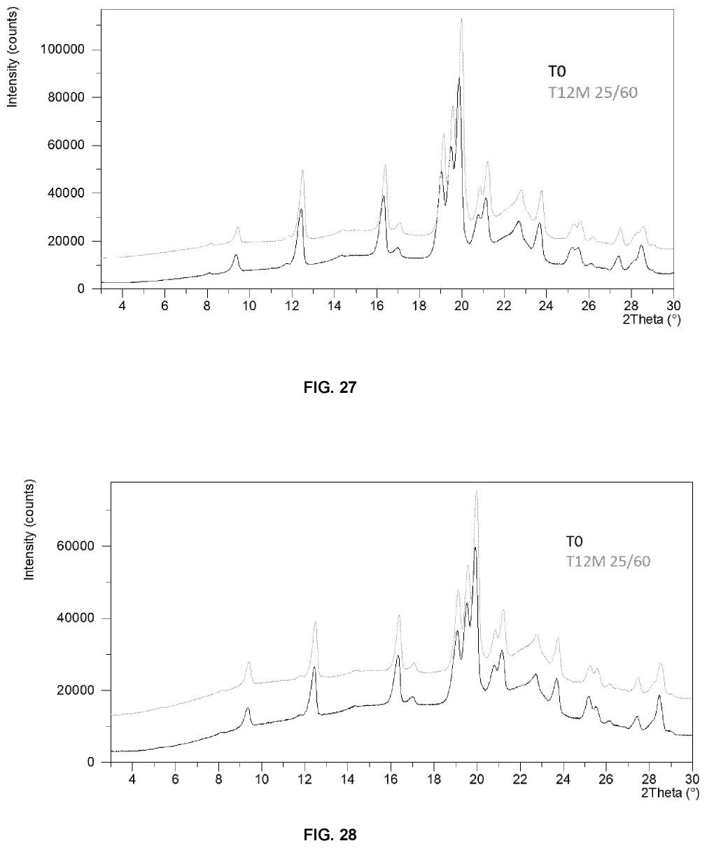

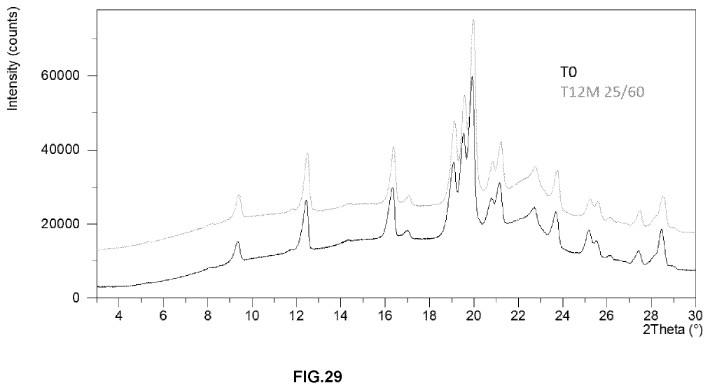

FIG. 27, FIG. 28 and FIG. 29 show respectively the X-Ray Powder Diffraction Pattern of Tablets A, B and D at before and after 12 months of storage at the conditions mentioned here above.

1. A solid dispersion comprising an amorphous compound of formula (I) and a polymer matrix.

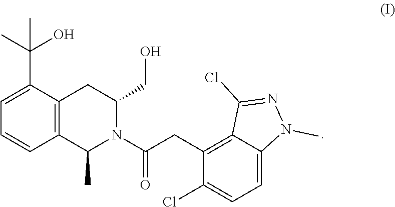 [SMILES: C[C@H]1c2cccc(C(C)(C)O)c2C[C@H](CO)N1C(=O)Cc1c(Cl)ccc2c1c(Cl)nn2C]

2. The solid dispersion according to claim 1 wherein the polymer matrix is selected from the group consisting of hydroxy propyl methyl cellulose acetate, co-polymer N-vinyl-2-pyrrolidone/vinyl acetate, polyvinyl pyrrolidone, hypromellose phthalate, and Hypromellose.

3. The solid dispersion according to claim 2 wherein the polymer matrix is hydroxy propyl methyl cellulose acetate or co-polymer N-vinyl-2-pyrrolidone/vinyl acetate.

4. The solid dispersion according to claim 1 which comprises about 30% to about 60% weight of the amorphous compound of formula (I) compared to total weight of amorphous solid dispersion.

5. The solid dispersion according to claim 4 which comprises about 40% weight of the amorphous compound of formula (I) compared to total weight of amorphous solid dispersion.

6. The solid dispersion according to claim 1 which has a glass transition temperature (Tg) greater than about 80° C.

7. The solid dispersion according to claim 6 which has a glass transition temperature (Tg) greater than about 100° C.

8. A process of preparation of a solid dispersion according to claim 1 which comprises the following steps:
(i) dissolving a monohydrated crystalline form of the compound of formula (I) and hydroxy propyl methyl cellulose acetate, co-polymer N-vinyl-2-pyrrolidone/vinyl acetate, polyvinyl pyrrolidone, hypromellose phthalate, or hypromellose in a solvent;
(ii) conveying the solution obtained as a result of step (i) into an atomization chamber;
(iii) contacting the droplets formed as a result of step (ii) with a hot drying gas;
(iv) evaporating the solvent;
(v) separating the solid dispersion obtained from the drying gas.

9. A process of preparation of a solid dispersion according to claim 1 which comprises the following steps:
(i) Mixing a monohydrated crystalline form of the compound of formula (I) and hydroxy propyl methyl cellulose acetate, co-polymer N-vinyl-2-pyrrolidone/vinyl acetate, polyvinyl pyrrolidone, hypromellose phthalate, or hypromellose;
(ii) Feeding the mixture obtained under step (i) into a hot melt extruder wherein a section made of barrel and screws conveys the mixture continuously at a temperature greater than 150° C. until a melt is obtained;
(iii) Cooling the melt obtained in step (ii) at ambient temperature.

10. A pharmaceutical composition comprising the solid dispersion according to claim 1 in association with one or more pharmaceutically acceptable excipients.

11. A pharmaceutical comprising a solid dispersion according to claim 1 which consists of a tablet comprising:
Between about 20% and about 60% per weight of amorphous solid dispersion;
Between about 10% and about 50% per weight of lactose monohydrate;
Between about 10% and about 50% per weight of microcrystalline cellulose;
Between about 1% and about 5% per weight of croscarmellose sodium;
Between about 0.1% and about 2% per weight of colloidal anhydrous silica; and
Between about 0.1% and about 5% per weight of magnesium stearate;
Compared to the total weight of the uncoated tablet.

12. (canceled)

13. (canceled)

14. A method of treatment of and/or prevention of cognitive and negative symptoms in schizophrenia, cognitive impairment related to neuroleptic therapy, Mild Cognitive impairment (MCI), impulsivity, Attention-Defficit Hyperactivity Disorder (ADHD), Parkinson's disease and other movement disorders, dystonia, Parkinson's dementia, Huntington's disease, dementia with Lewy Body, Alzheimer's disease drug addiction, sleep disorders, apathy, traumatic spinal cord injury or neuropathic pain which comprises administering to a patient in need of such treatment an effective amount of an amorphous solid dispersion according to claim 1.

15. A method of treatment of and/or prevention of cognitive and negative symptoms in schizophrenia, cognitive impairment related to neuroleptic therapy, Mild Cognitive impairment (MCI), impulsivity, Attention-Defficit Hyperactivity Disorder (ADHD), Parkinson's disease and other movement disorders, dystonia, Parkinson's dementia, Huntington's disease, dementia with Lewy Body, Alzheimer's disease drug addiction, sleep disorders, apathy, traumatic spinal cord injury or neuropathic pain which comprises administering to a patient in need of such treatment an effective amount of a pharmaceutical composition according to claim 10.

16. A solid dispersion comprising amorphous 2-(3,5-dichloro-1-methyl-indazol-4-yl)-1-[(1S,3R)-3-(hydroxymethyl)-5-(1-hydroxy-1-methyl-ethyl)-1-methyl-3,4-dihydro-1H-isoquinolin-2-yl]ethanone and a polymer matrix.
